(19) United States
(12) Patent Application Publication
(10) Pub. No.: US 2026/0334941 A1
(43) Pub. Date: Oct. 29, 2026

(54) SUTURE FASTENENING SYSTEMS AND METHODS

(71) Applicant: cLoc Medical Inc., Maple Grove, MN (US)

(72) Inventors: Benjamin Montag, Delano, MN (US); Gregory Gabay, New Hope, MN (US); Aashiish Agnihotri, Maple Grove, MN (US); Lucas Haarstad, Columbia Heights, MN (US); Jeffrey Sandstrom, Scandia, MN (US); Trevor Laughlin, Medina, MN (US); Caleb Vainikka, St Michael, MN (US)

(21) Appl. No.: 19/731,976

(22) Filed: Jul. 6, 2026

Related U.S. Application Data
(63) Continuation-in-part of application No. 19/249,585, filed on Jun. 25, 2025.
Application No. 19/249,585 is a division of application No. 19/035,170, filed on Jan. 23, 2025, now Pat. No. 12,369,906.
(60) Provisional application No. 63/742,242, filed on Jan. 6, 2025.
(60) Provisional application No. 63/839,690, filed on Jul. 7, 2025.
(60) Provisional application No. 63/907,881, filed on Oct. 29, 2025.
(60) Provisional application No. 64/044,908, filed on Apr. 20, 2026.

(51) Int. Cl. A61B 17/04

(52) U.S. Cl. CPC A61B 17/0469; A61B 17/0483; A61B 2017/0479

(57) ABSTRACT

A suture fastener delivery device is configured to capture a suture and deploy deformable suture fasteners to secure the suture within a surgical field. The delivery device includes a shaft extending between a controller and a distal tip, reserve suture fasteners within the shaft, and an indexing mechanism for sequentially advancing the reserve suture fasteners to the distal tip. The distal tip includes a suture slot for capturing the suture and crimping jaws for deforming the suture fastener to secure the suture. In operation the delivery device can, via a controller, load a suture fastener at the distal tip, partially crimp the suture fastener about a suture, fully crimp the suture fastener about the suture, and cut the excess suture strands. The device can also include a counter configured to indicate a quantity of remaining suture fasteners, and a loader configured to load additional suture fasteners into the delivery device.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Surgical sutures can be secured via hand tied knots, instrument tied knots, or by deforming a malleable material around strands of the suture, thereby securing it. Instruments which allow deployment of such malleable material must be precise and intuitive for a clinician to use in order to ensure that the sutures are appropriately secured. Such instruments can present challenges to a clinician such as difficulties in making appropriate contact with sutures to be secured, and continually moving out of the sterile surgical field to obtain new suture fasteners to secure additional sutures. Therefore, improvements in instruments relating to deployment of suture fasteners, and suture fasteners themselves as well as methods for using such instruments are desirable.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

In one or more embodiments, a suture fastener delivery device comprises a controller positioned on a proximal end of the suture fastener delivery device, the controller comprising at least one control element, a fastener delivery end positioned on a distal end of the suture fastener delivery device, the fastener delivery end comprising a suture fastener configured to be deployed to secure a suture and crimping jaws positioned about an outer surface of the suture fastener, and a shaft extending between the controller and the fastener delivery end from the proximal end to the distal end of the suture fastener delivery device. The shaft comprises a rail extending along an interior length of the shaft, a plurality of reserve suture fasteners mounted along the rail, a side suture slot positioned about a side of the shaft, the side suture slot configured to allow the suture to exit the suture fastener delivery device, and a suture cutting blade, the suture cutting blade positioned adjacent to the rail within the shaft.

In one or more embodiments, a suture fastener delivery device includes a controller positioned on a proximal end of the suture fastener delivery device and a fastener delivery end positioned on a distal end of the suture fastener delivery device. The controller includes a control element, and the fastener delivery end includes a suture fastener configured to secure a suture and one or more crimping jaws configured to deform the suture fastener. A shaft extends between the controller and the fastener delivery end and includes a rail extending along an interior length of the shaft, a plurality of reserve suture fasteners mounted along the rail, a side suture slot configured to receive a suture, and a suture cutting blade configured to sever excess suture material.

In some embodiments, the suture fastener has a substantially C-shaped cross section and includes a collar extending circumferentially about an outer surface of the suture fastener.

In some embodiments, the collar includes a discontinuity co the discontinuity comprising a gap extending through the collar.

In some embodiments, the discontinuity is positioned opposite the suture receiving opening of the suture fastener.

In some embodiments, the collar includes a protrusion extending into the suture receiving opening and configured to retain a suture within the suture fastener.

In some embodiments, the collar includes a groove configured to position a suture at a preferred position within the suture fastener.

In some embodiments, the groove comprises a partial indentation positioned adjacent to the suture receiving opening.

In some embodiments, an inner diameter of the suture fastener includes one or more frictional elements configured to increase retention of the suture after crimping.

In some embodiments, actuation of the control element to a first actuation position advances a distal-most reserve suture fastener to the fastener delivery end.

In some embodiments, the fastener delivery end includes a distal tip formed from stainless steel.

In some embodiments, the crimping jaws are configured to impart a crimping force at a location offset from a central axis of the distal tip.

In some embodiments, the crimping jaws include a floating jaw positioned adjacent to the fastener delivery end.

In some embodiments, the delivery device further comprises a jaw came, the jaw cam comprising a contact surface and the jaw cam configured to engage with the floating jaw at the contact surface to impart a crimping force.

In some embodiments, the jaw cam includes an angled contact surface configured to engage a corresponding contact surface of the floating jaw.

In one or more embodiments, a method of installing a suture fastener within a surgical field includes advancing a suture fastener from a stack of reserve suture fasteners housed within a shaft of the suture fastener delivery device to a distal end of the shaft such that an opening of the suture fastener is aligned with a side opening of the distal end of the shaft, positioning the suture fastener delivery device such that the side opening of the distal end and the opening of the suture fastener are adjacent to a suture, capturing the suture within the suture fastener such that the suture rests within a body of the suture fastener, and exits through a portion of the side suture slot curving about the circumference of the distal end of the suture fastener delivery device, partially deforming the suture fastener via a crimping mechanism such that the suture is restricted from exiting the suture fastener device via the opening of the suture fastener, sliding the distal end of the suture fastening device along the suture until the suture fastener is positioned at a fastening site, further deforming the suture fastener via the crimping mechanism such that the suture is secured, and cutting a trailing end of the suture via the suture cutting blade.

In some embodiments, advancing the suture fastener includes actuating a control element to a first actuation position.

In some embodiments, partially deforming the suture fastener includes actuating the control element to a second actuation position.

In some embodiments, further deforming the suture fastener includes actuating the control element to a third actuation position.

In some embodiments, the control element is movable between a plurality of actuation positions corresponding to different operational stages of the suture fastener delivery device, and the control element engages a stop position at each actuation position to provide tactile feedback to a user.

In some embodiments, the suture fastener transitions from a substantially C-shaped configuration to a substantially closed tubular configuration during after partial deformation of the suture fastener.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the disclosure will be better understood from the following description taken in conjunction with the accompanying Figures, in which:

FIG. 1 is a perspective view of a suture fastener delivery device according to one embodiment of the present disclosure.

FIG. 2 is a perspective view of a suture fastener according to one embodiment of the present disclosure.

FIGS. 3A-3C depict perspective views of the suture fastener according to various other embodiments of the present disclosure.

FIG. 4A-4C depict perspective views of the suture fastener according to further embodiments of the present disclosure.

FIG. 5 is a side view of a shaft and distal tip of the suture fastener delivery device, depicting the internal components.

FIGS. 6A-6C illustrate a rail having a friction region for retaining and positioning suture fasteners during delivery according to one embodiment of the present disclosure.

FIGS. 7A-7C illustrate an indexing mechanism configured to advance successive suture fasteners toward a distal end of the delivery device.

FIG. 8 illustrate a distal tip arrangement according to one embodiment of the present disclosure.

FIG. 9 illustrates a crimping mechanism for deforming the suture fasteners according to one embodiment of the present disclosure.

FIGS. 10A-10B illustrate a cutting mechanism including a cutting blade and a counter-blade arrangement according to one embodiment of the present disclosure.

FIGS. 11A-11D illustrate one embodiment of a sequence of operation of the suture fastener delivery device.

FIG. 12 illustrates a method flowchart of one embodiment of a method for loading a suture into the suture fastener via the suture fastener delivery device.

FIG. 13 illustrates a method flowchart of one embodiment of a method for securing the a suture via the suture fastener delivery device.

FIGS. 14A-14D illustrate one embodiment of actuation positions of a control mechanism in the sequence of operation of the suture fastener delivery device.

FIGS. 15A-15B illustrate various embodiments of a counter configured to indicate a quantity of suture fasteners remaining within the delivery device.

FIGS. 16A-16B illustrate a single-use loader configured to load a suture fastener into a distal end of the delivery device.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, and/or components have not been described in detail so as not to obscure the discussion.

The present disclosure describes a novel and advantageous suture fastener delivery device as well as a novel and advantageous suture fastener. The suture fastener delivery device includes a handle, a robotic control arm, or a similar device on a proximal end (i.e., proximal to the user) a fastener delivery mechanism on the distal end (i.e., distal from the user), and a shaft extending between the proximal end and the distal end. The handle comprises a control element for using the suture fastener delivery device. The shaft houses a rail having a stack of reserve suture fasteners mounted on the rail and an indexing mechanism for advancing reserve suture fasteners forward for deployment. The fastener delivery mechanism includes a suture fastener to be deployed, a side suture slot for receiving sutures, crimping jaws positioned about the suture fastener to secure a suture within the suture fastener, and a suture cutting blade for cutting sutures once secured.

In operation, a first activation of the control mechanism positions a suture fastener in a deployment position at the distal end. The suture fastener delivery device can then be maneuvered such that a suture is captured within the suture fastener and the trailing ends of the suture extend through the side slot in the shaft of the delivery device. Upon a second activation of the control mechanism, the suture fastener delivery device can activate the crimping jaws to partially deform the suture fastener, thereby securing the suture laterally within the suture fastener, while still permitting movement of the suture fastener along the length of the suture. The suture fastener delivery device can then be maneuvered such that the suture fastener is proximate to a suture site. Upon a third activation of the control mechanism, the suture fastener delivery device can further activate the crimping jaws to fully deform the suture fastener, thereby securely entrapping the suture within the suture fastener and the suture fastener delivery device can deploy the cutting mechanism to cut the excess strands of the suture exiting from the side slot in the shaft. After the third activation of the control mechanism, the control mechanism is reset, allowing for a new first activation to deploy a reserve suture fastener from the stack of reserve suture fasteners. This allows for the suture fastener delivery device to apply multiple suture fasteners (e.g., 10, 16, 20, or more than 20 suture fasteners) without removing the suture fastener delivery device from the surgical field.

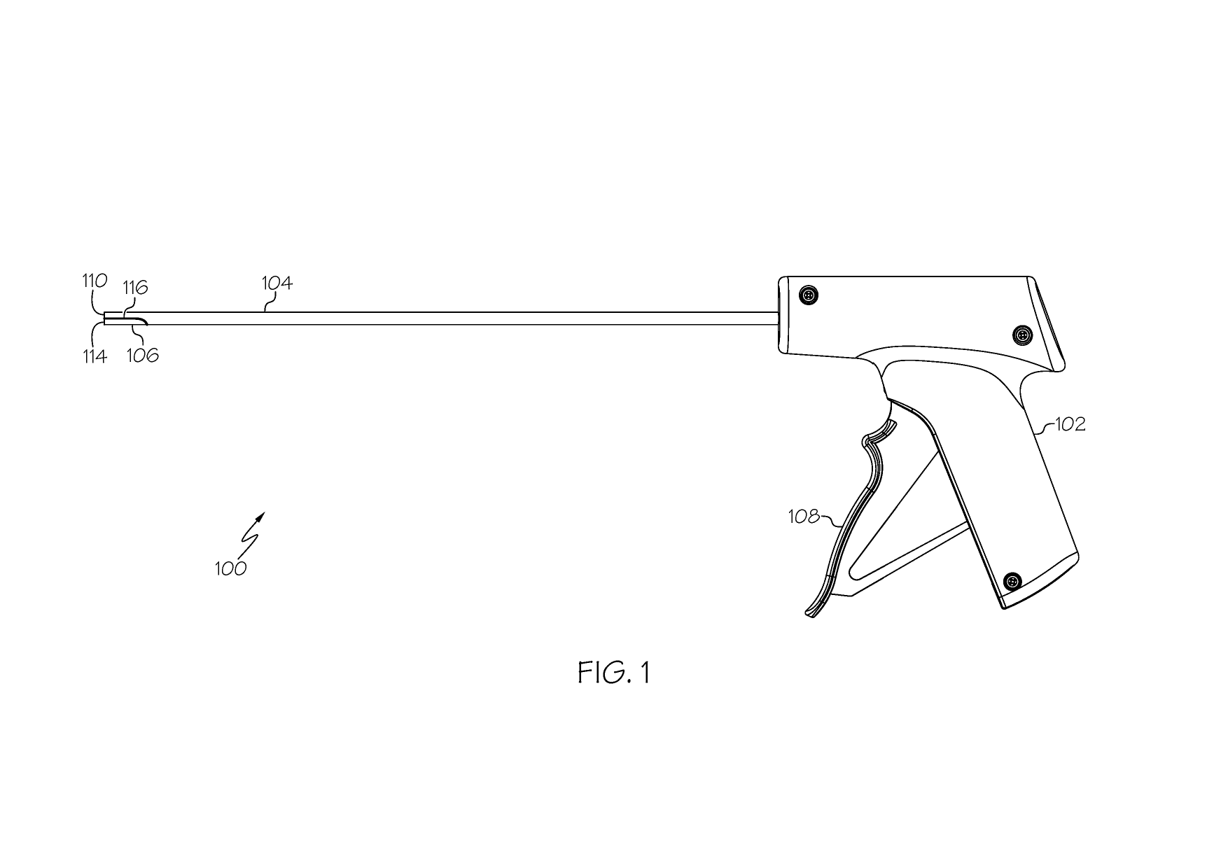

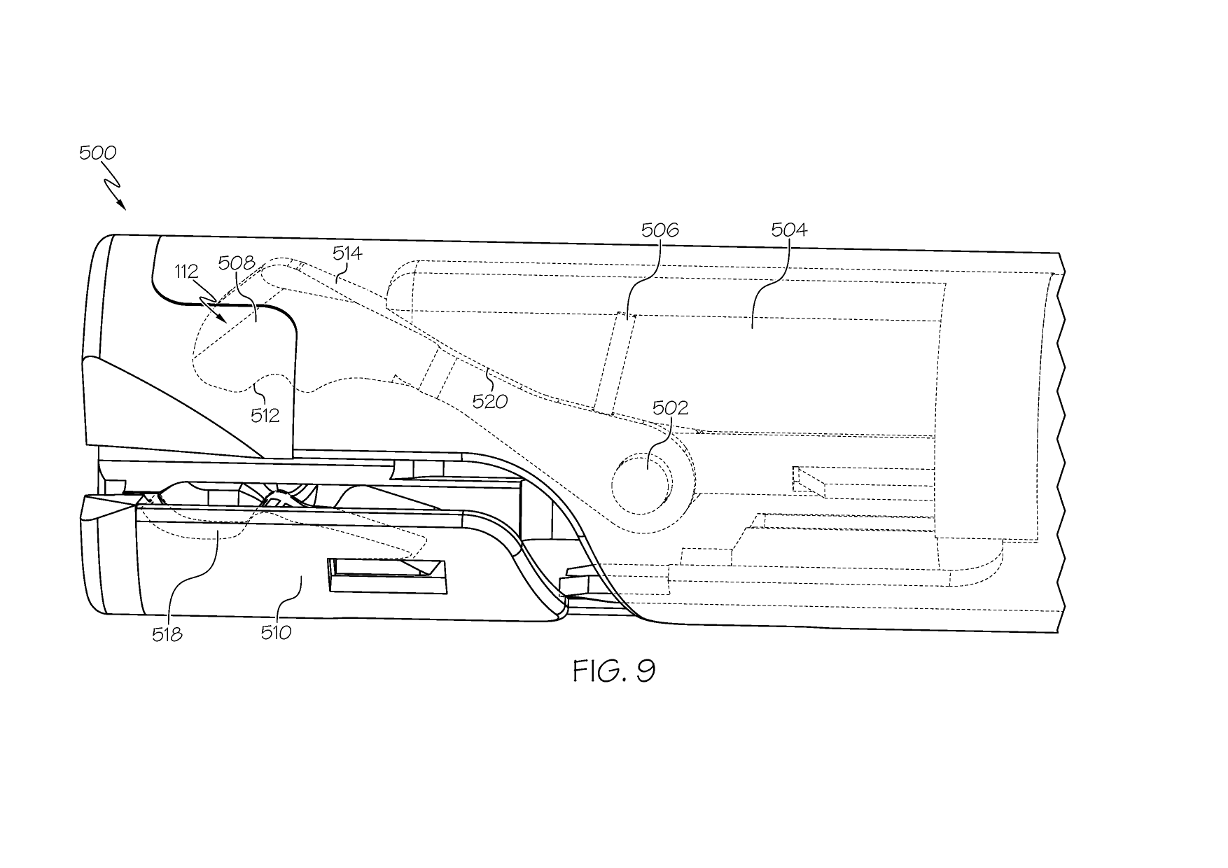

Referring to FIG. 1, a suture fastener delivery device 100 is illustrated according to one embodiment of the present disclosure. Suture fastener delivery device 100 generally includes controller 102 positioned at a proximal end of the device 100, fastener delivery end 106, and shaft 104 extending between controller 102 and fastener delivery end 106. Controller 102 can include one or more control elements 108, such as a trigger, button, lever, slider, or other user-actuatable mechanism configured to operate one or more functions of suture fastener delivery device 100. Shaft 104 may define an elongated body configured to house one or more internal components including a plurality of reserve suture fasteners, an indexing mechanism, a crimping mechanism, and a cutting mechanism. Fastener delivery end 106 may include distal tip 110, one or more crimping jaws 112 (best depicted in FIGS. 8 and 9), and suture fastener 114 positioned for deployment onto a suture. Shaft 104 further includes side suture slot 116 extending along at least a portion of shaft 104 and configured to permit lateral introduction and routing of a suture relative to suture fastener 114. In some embodiments, side suture slot 116 includes a curved portion, curving about the circumference of shaft 104.

Controller 102 is operably coupled to fastener delivery end 106 through one or more actuation members extending through shaft 104. In operation, actuation of control element 108 causes a reserve suture fastener to be advanced toward and placed in a deployment position at fastener delivery end 106, actuates crimping jaws 112 to deform suture fastener 114, and/or actuates a cutting mechanism to sever excess suture material. As described in greater detail below, suture fastener delivery device 100 is configured to permit a surgeon to position a suture fastener 114 at distal tip 110 for deployment, load a suture into suture fastener 114, partially secure the suture within suture fastener 114 through a partial crimping operation, reposition suture fastener 114 along the suture, fully crimp suture fastener 114, and cut excess suture material.

The arrangement of controller 102, shaft 104, and fastener delivery end 106 provides a compact delivery system capable of sequentially deploying multiple suture fasteners while facilitating controlled placement, tensioning, fastening, and cutting operations within a surgical field. Subsequent figures illustrate various embodiments of the suture fastener 114, the crimping mechanisms, the cutting mechanisms, the indexing mechanisms, and associated loading systems in greater detail.

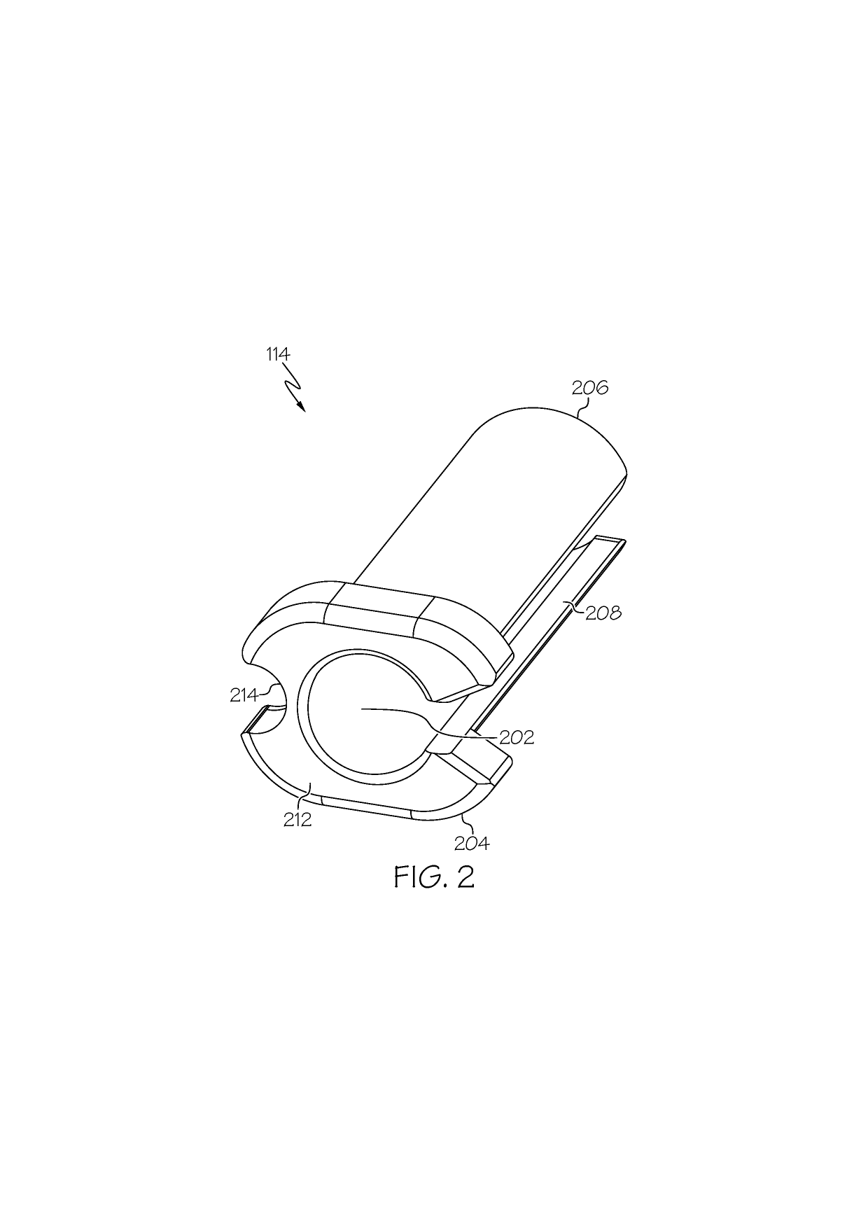

Referring to FIG. 2, a suture fastener 114 is illustrated according to one embodiment of the present disclosure. Suture fastener 114 generally includes body 202 extending between first end 204 and second end 206 and defining a longitudinal suture receiving opening 208 configured to receive a suture therein. Body 202 has a substantially C-shaped cross section such that a suture received through suture receiving opening 208 rests within body 202. Collar 212 extends circumferentially about at least a portion of body 202 adjacent first end 204. Collar 212 includes discontinuity 214, illustrated in FIG. 2 as a gap extending through collar 212. In the illustrated embodiment, discontinuity 214 is positioned substantially opposite suture receiving opening 208.

Collar 212 is integrally formed with body 202 and is configured to deform together with body 202 during crimping of the suture fastener 114. During operation of suture fastener delivery device 100, a suture may be laterally introduced into the suture receiving opening 208 such that the suture rests within body 202, and suture fastener 114 may subsequently be deformed from an open configuration toward a closed configuration. In some embodiments suture fastener 114 may first be deformed into a partially crimped position such that suture receiving opening 208 is closed and suture fastener 114 is substantially tubular, but the suture is still free to move within body 202. In such an embodiment, suture fastener 114 can then be further deformed such that the suture is securely retained within body 202, without the ability to move. Discontinuity 214 functions as a force-relief feature that permits localized deformation of collar 212 during closure of suture receiving opening 208. As a result, the force required to deform suture fastener 114 may be reduced while maintaining adequate retention strength after crimping.

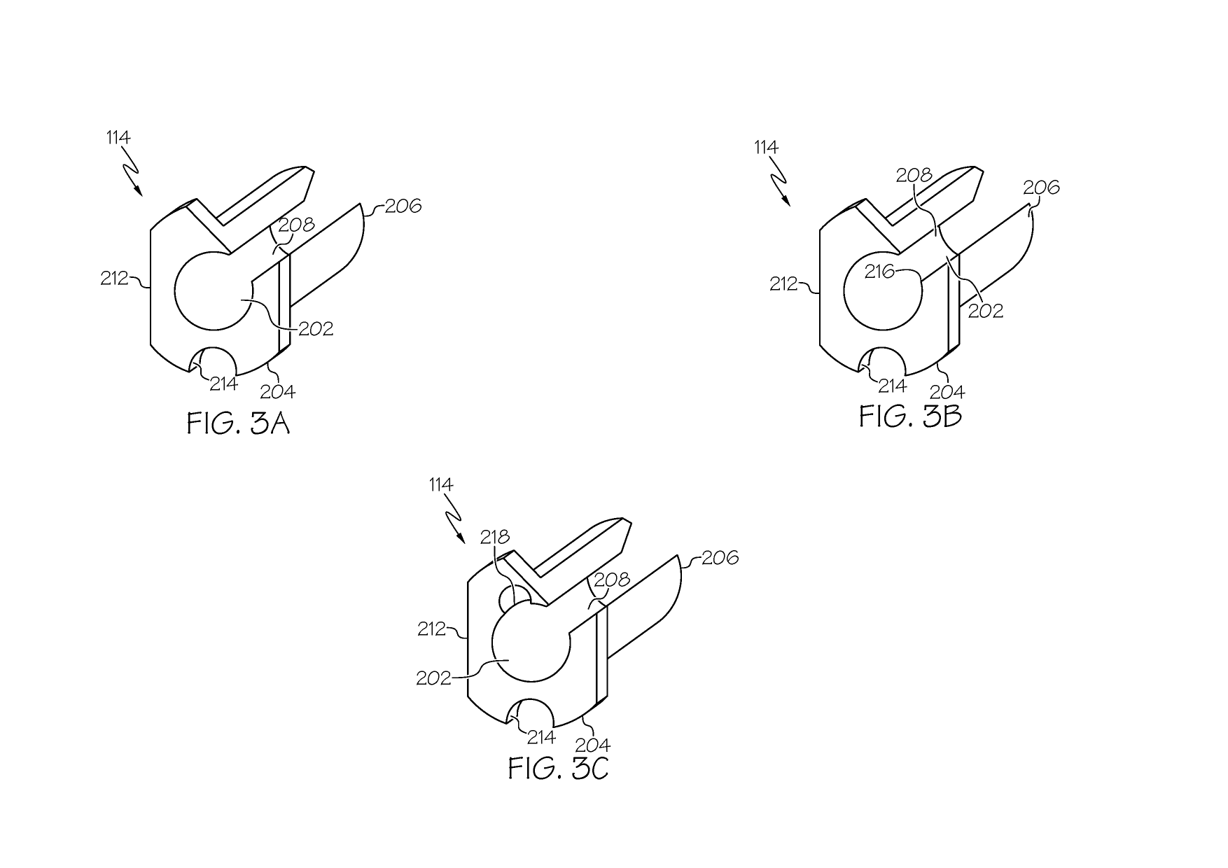

FIGS. 3A-3C illustrate additional embodiments of suture fastener 114. In FIG. 3A, discontinuity 214 is positioned at a location offset from a position directly opposite suture receiving opening 208. Similar to the embodiment of FIG. 2, discontinuity 214 permits deformation of collar 212 during crimping while acting as a force-relief feature.

Referring to FIG. 3B, suture fastener 114 further includes suture retention feature 216. Suture retention feature 216 is positioned adjacent to suture receiving opening 208 and comprises a protrusion extending inwardly from collar 212 toward suture receiving opening 208. Suture retention feature 216 is configured to permit the lateral passage of the suture through suture receiving opening 208 and into body 202, but to restrict the suture from laterally exiting suture fastener 114 through suture receiving opening 208.

Referring to FIG. 3C, suture fastener 114 includes suture positioning feature 218. In the illustrated embodiment, suture positioning feature 218 comprises a groove, recess, indentation, or other localized surface feature formed in collar 212. Suture positioning feature 218 is configured to guide a suture through suture receiving opening 208 toward a preferred location within body 202 during crimping. By encouraging repeatable positioning of the suture within suture fastener 114, suture positioning feature 218 can improve consistency of crimping forces and retention performance across multiple deployments.

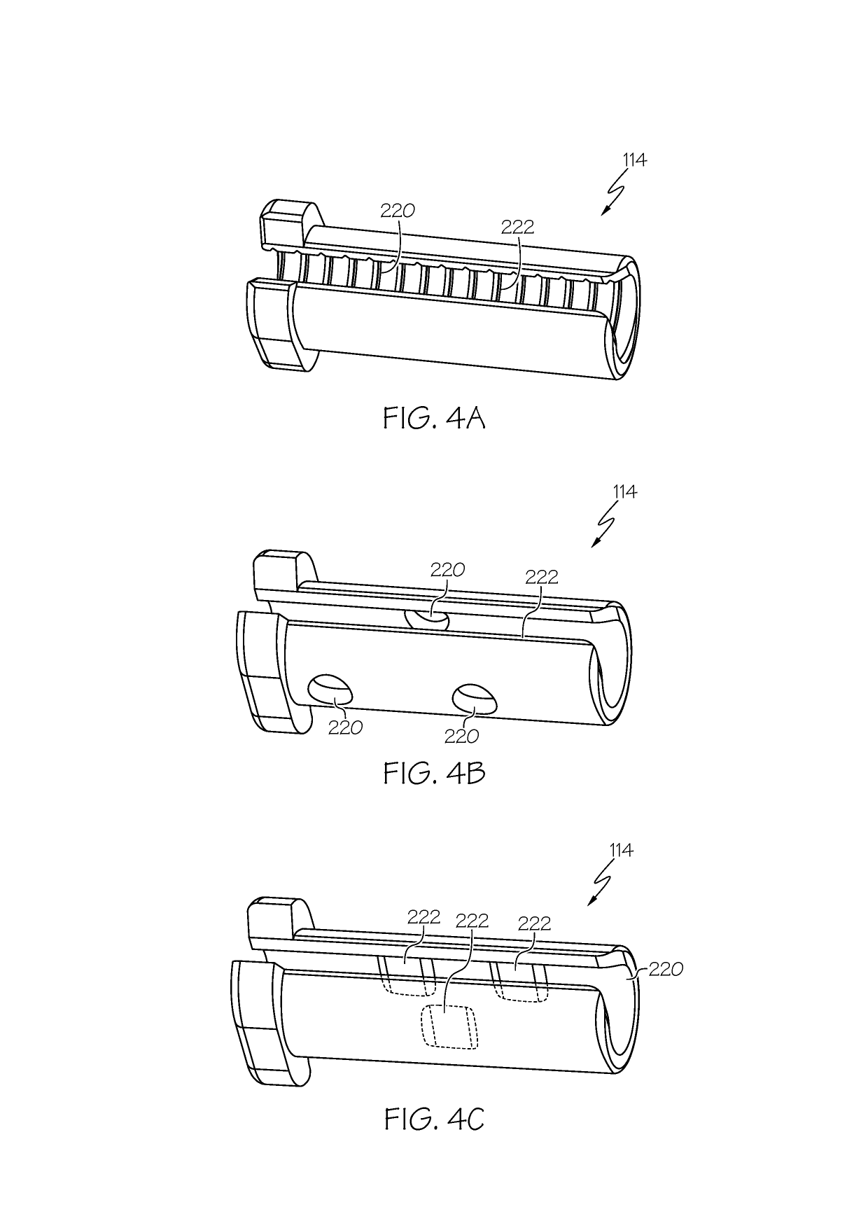

FIGS. 4A-4C illustrate additional embodiments of suture fastener 114 including one or more frictional elements 220 positioned on an interior surface 222 of body 202. Frictional elements 220 may comprise ridges, holes, protrusions, serrations, knurls, textured regions, dimples, teeth, surface roughening features, or combinations thereof. Frictional elements 220 are positioned to engage the suture disposed within suture receiving opening 208 after the crimping of suture fastener 114. During deformation of suture fastener 114, frictional elements 220 cooperate with opposing portions of body 202 to increase frictional engagement between suture fastener 114 and the suture. Accordingly, frictional elements 220 may increase resistance to slippage of the suture relative to the suture fastener 114 and improve the overall holding strength of the deployed fastener.

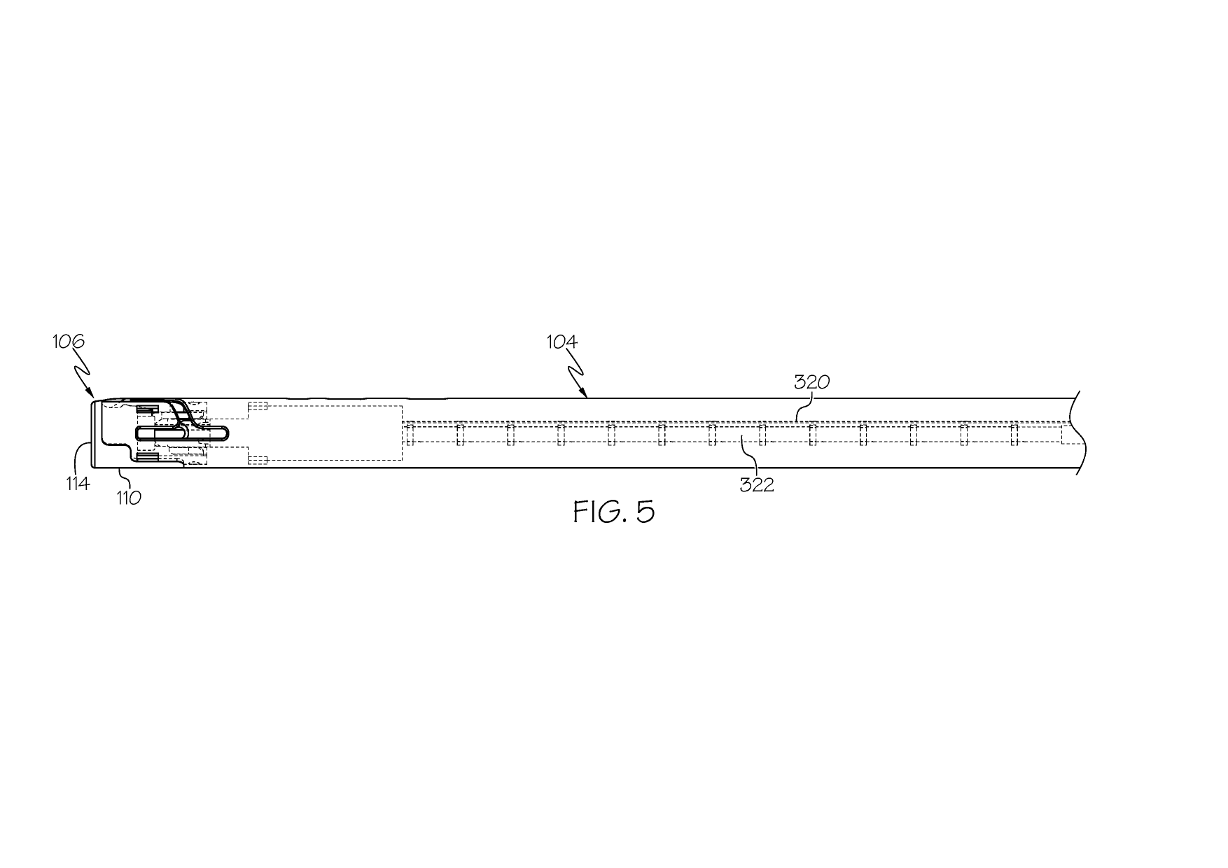

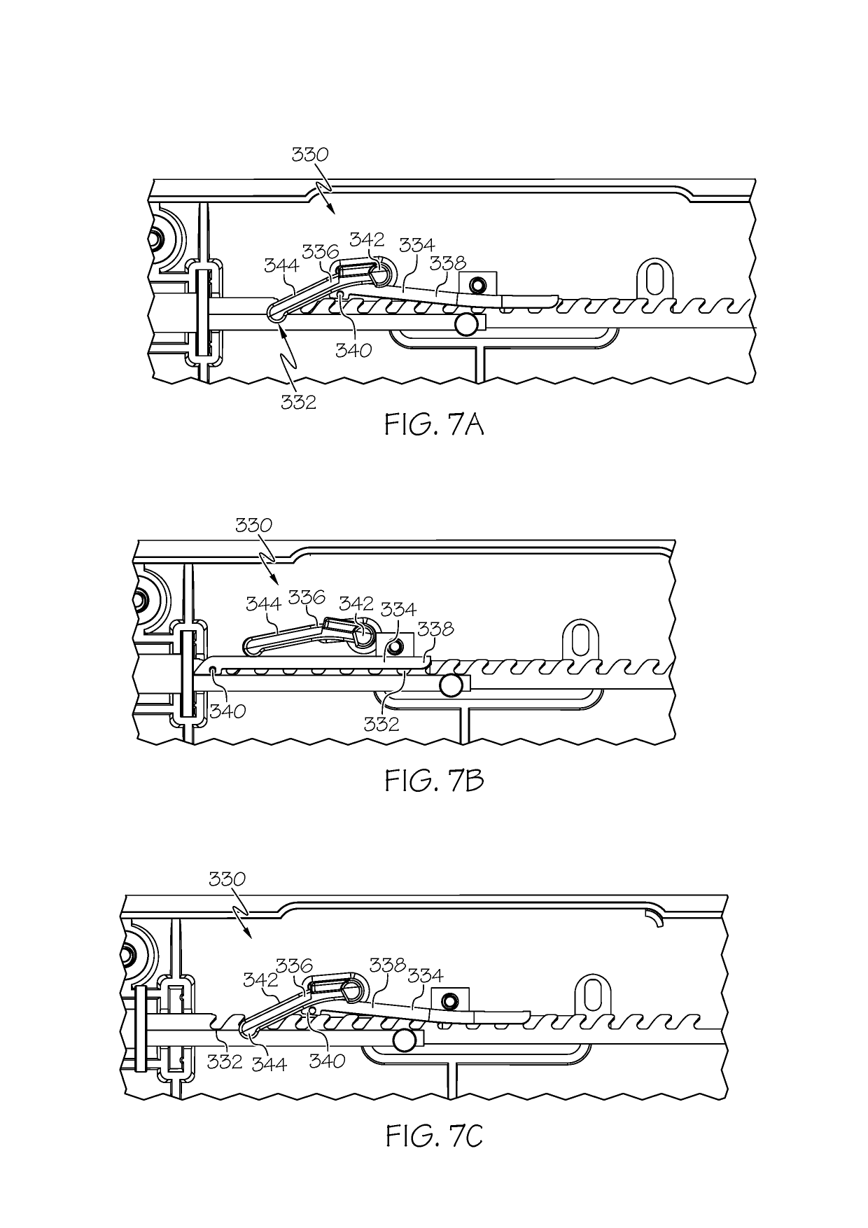

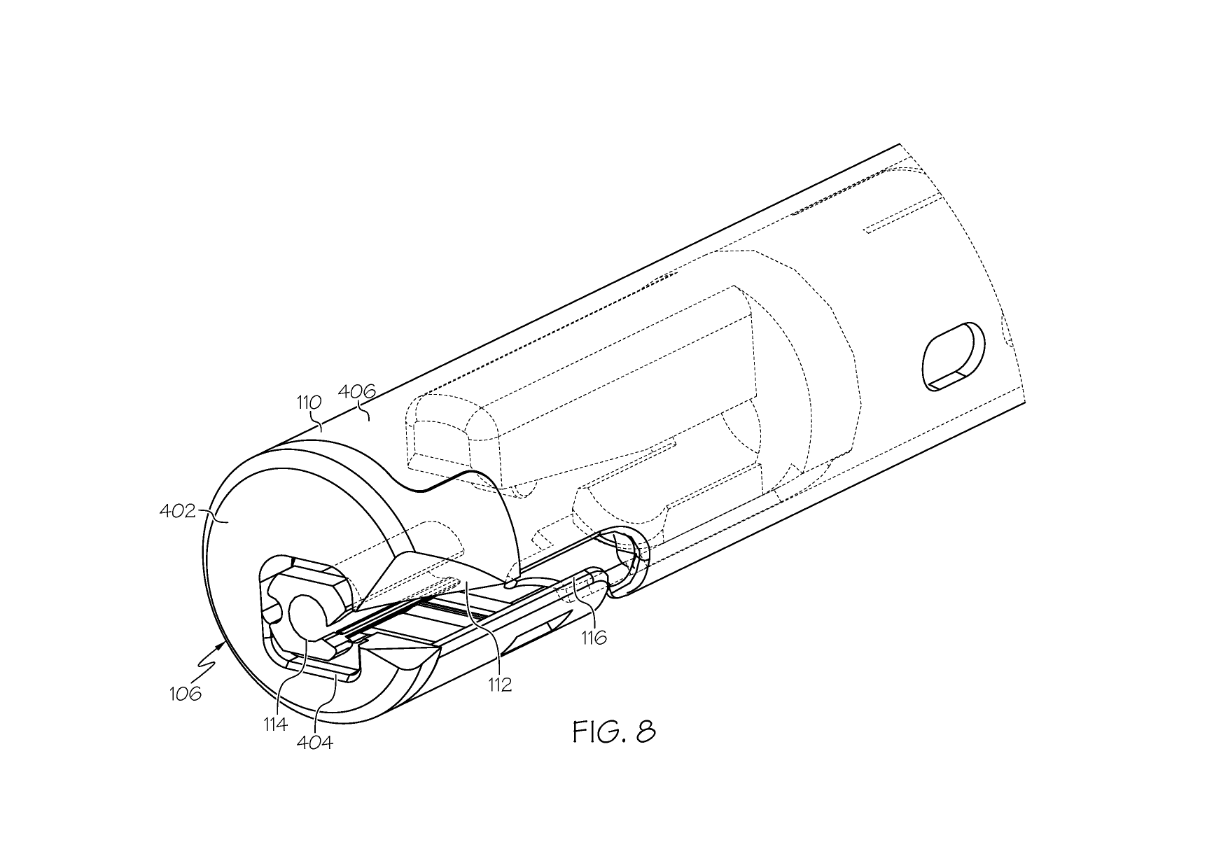

Referring to FIG. 5, shaft 104 and fastener delivery end 106 of suture fastener delivery device 100 are illustrated according to one embodiment of the present disclosure. Shaft 104 extends between controller 102 and fastener delivery end 106 and defines an interior passage configured to house a plurality of reserve suture fasteners 322, and associated indexing mechanism, and an associated delivery components. The indexing mechanism is described in more detail in the description of FIGS. 7A-7C, and the delivery components are described in more detail in the description of FIG. 8.

In the illustrated embodiment, rail 320 extends longitudinally through at least a portion of shaft 104 and is configured to support reserve suture fasteners 322 in a stacked arrangement. Fastener delivery end 106 is positioned at a distal end of shaft 104 and includes distal tip 110 configured to receive and deploy individual suture fasteners 114. Shaft 104 thereby functions as a storage and delivery assembly capable of housing multiple reserve suture fasteners 322 and sequentially delivering reserve suture fasteners 322 to fastener delivery end 106 during a surgical procedure. By storing a plurality of reserve suture fasteners 322 within shaft 104, suture fastener delivery device 100 may perform multiple fastening operations without requiring removal of the device from the surgical field for reloading.

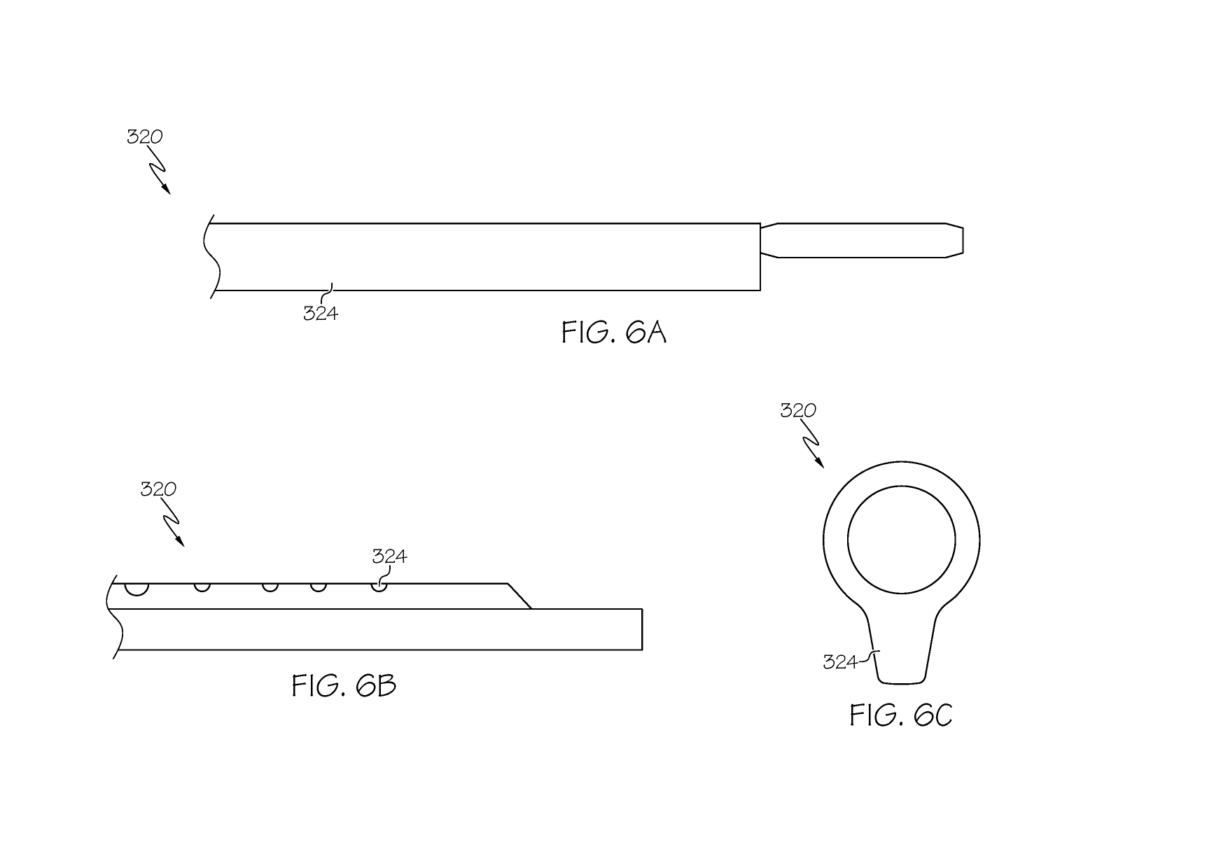

Referring to FIGS. 6A-6C, in some embodiments, rail 320 includes friction region 324 adjacent a distal portion of rail 320. Friction region 324 is configured to create an interference fit or frictional engagement with reserve suture fasteners 322 as reserve suture fasteners 322 are advanced toward fastener delivery end 106. The friction regions can prevent inadvertent distal migration or release of reserve suture fasteners 322 and can further ensure that a distal-most reserve suture fastener is pulled tightly against distal tip 110 during retraction of indexing mechanism 330. The friction regions may be formed by localized deformation of the rail, including crimped, swaged, expanded, roughened, melted, textured, enlarged-diameter, tapered, or otherwise interference-producing portions of the rail. In some embodiments, the rail comprises a metallic rail and the friction region is formed by swaging or crimping the rail. In other embodiments, the rail comprises a polymeric rail and the friction region is formed by localized heating, melting, expansion, or deformation of the rail. In still other embodiments, a hollow plastic rail component can be loaded with an oversized and/or tapered mandrel to expand its outer diameter to create interference in areas where friction is desired. Example friction regions 324 are depicted in FIGS. 6A-6C.

Referring to FIGS. 7A-7C, indexing mechanism 330 positioned within shaft 104 and is illustrated according to one embodiment of the present disclosure. FIGS. 7A-7C further illustrate the sequential steps taken by indexing mechanism 330 to advance a suture fastener to a deployment position.

Indexing mechanism 330 is configured to advance a distal most reserve suture fastener of reserve suture fasteners 322 to distal tip 110 by pushing the stack of reserve suture fasteners 322 along rail 320 toward fastener delivery end 106. The distal most reserve suture fastener is thus placed in a deployment position as suture fastener 114. Indexing mechanism 330 can include slotted tube 332, pusher 334, pin 336, and one or more associated actuation components operably coupled to the control element 108. Pusher 334 includes pusher arm 338 and pusher head 340. Pin 336 includes fixed portion 342 and deflectable portion 344.

Reserve suture fastener 114 are housed within slotted tube 332. Pusher arm 338 extends longitudinally from control element 108 along shaft 104 toward fastener delivery end 106. Pusher head 340 is positioned on a distal end of pusher arm. Pusher head 340 is shaped to engage within a slot of slotted tube 332. Pusher head 340 can be any shape or structure suitable for engagement with slotted tube 332, including a pin, tab, protrusion, hook, or other suitable engagement structure configured to transfer force from pusher arm 338 to slotted tube 332. Fixed portion 342 of pin 336 is positioned radially above slotted tube 332 and pusher 334. Deflectable portion 344 of pin 336 extends downward to engage within a slot of slotted tube 332. Deflectable portion 344 of pin 336 engages slotted tube 332 within a slot that is closer to fastener delivery end 106 than the slot of slotted tube 332 which is engaged by pusher head 340 of pusher 334. Though reference is made to slots within slotted tube 332, it is understood that any number of suitable alternatives can be used (e.g., recesses, detents, openings, notches, or other engagement features),

In operation, actuation of control element 108 causes indexing mechanism 330 to advance a distal-most reserve suture fastener 114 toward fastener delivery end 106. Referring to FIG. 7A, in an initial state, deflectable portion 344 of pin 336 is engaged with a slot of slotted tube 332. Pusher head 340 of pusher 334 is positioned atop a slot of slotted tube 332, and is ready to be engaged with such slot upon activation of control element 108. In the depicted embodiment, there exists a gap of one slot between the slot in which pin 336 is engaged and the slot in which pusher 334 is preparing to engage.

Referring to FIG. 7B, during a forward stroke of control element 108, pusher arm 338 and pusher head 340 are advanced distally. Pusher head 340 engages a slot of slotted tube 332 and pushes slotted tube 332 distally along rail 320. Accordingly, the reserve suture fasteners 322 are also advanced toward fastener delivery end 106. The distal advancement of slotted tube 332 continues until the distal most suture fastener of reserve suture fasteners 322 is advanced to a deployment position.

Still referring to FIG. 7B, as slotted tube 332 moves distally during the forward stroke, deflectable portion 344 of pin 336, which is initially engaged with a slot of slotted tube 332, is displaced from such an engagement. In the depicted embodiment, the slots of slotted tube 332 are angled and deflectable portion 344 is correspondingly angled in order to allow for deflectable portion 344 to smoothly disengage from the slot when slotted tube 332 is advancing distally. Pin 336 elastically deflects and exits the slot, allowing slotted tube 332 to continue moving relative to pin 336. Following disengagement from the slot, the deflectable portion of pin 336 rides along an outer surface of slotted tube 332 while the slotted tube 332 continues its distal travel. Thus, pin 336 does not reengage a slot of slotted tube 332 while slotted tube 332 is pushed distally by pusher 334.

Referring to FIG. 7C, after the distal most suture fastener of reserve suture fasteners 322 is advanced to the deployment position, and upon release of control element 108, pusher 334 begins the return stroke, bringing slotted tube 332 proximally back toward controller 102. As slotted tube 332 is returned toward the control element, deflectable portion 344 of pin 336 engages with the next available slot of slotted tube 332. In the depicted embodiment, pin 336, which was originally engaged with a first slot of slotted tube 332, is engaged with a second slot of slotted tube 332 upon retraction. Thus, slotted tube 332 does not fully retract to its original position, thereby allowing for the next suture fastener 114 in the stack of reserve suture fasteners 322 to be appropriately distally positioned for deployment upon the next cycle of use of indexing mechanism 330. Pusher 334 is fully retracted such that pusher head 340 is positioned above the next available slot in the slotted tube. In the depicted embodiment, pusher 334, which was originally engaged with a third slot of slotted tube 332, is engaged with a fourth slot of slotted tube 332 upon retraction.

Accordingly, in the described embodiment, each complete advancement cycle of indexing mechanism 330 results in a net distal advancement of slotted tube 332, and hence of reserve suture fasteners 322, by approximately the length of one suture fastener 114. Indexing mechanism 330 thereby permits sequential positioning of individual reserve suture fasteners 322 at fastener delivery end 106 while maintaining the remaining reserve suture fasteners 322 within shaft 104. In this manner, indexing mechanism 330 provides controlled, repeatable, and incremental advancement of reserve suture fasteners 322 and facilitates successive deployment operations without requiring manual reloading of the suture fastener delivery device 100.

Referring to FIG. 8, fastener delivery end 106 of suture fastener delivery device 100 is illustrated in greater detail. Fastener delivery end 106 includes distal tip 110. Distal tip 110 includes distal tip face 402, suture fastener deployment opening 404, reinforced region 406, and side suture slot 116. Distal tip 110 also houses crimping jaws 112. Suture fastener deployment opening 404 is an opening in distal tip face 402. In some embodiments, suture fastener deployment opening 404 is positioned off-center with respect to a radial center of distal tip face 402. Reinforced region 406 is a portion of distal tip 110 which is substantially encompassed by a reinforced material. Reinforced region 406 is not tubular to allow for the side suture slot 116 to remain unblocked. In some embodiments the reinforced material is 17-4PH H900 stainless steel. Side suture slot 116 extends longitudinally along at least a portion of distal tip 110 from distal tip face 402. Side suture slot 116 curves circumferentially about at least a portion of distal tip 110

In operation, indexing mechanism 330 advances the distal-most reserve suture fastener of reserve suture fasteners 322 to suture fastener deployment opening 404. The distal most reserve suture fastener is thus placed in a deployment position as suture fastener 114. As suture fastener 114 enters suture fastener deployment opening 404, suture fastener 114 passes between crimping jaws 112. In some embodiments, crimping jaws 112 define a spacing configured to create a frictional engagement or interference fit with suture fastener 114. The frictional engagement may be sufficient to retain suture fastener 114 within suture fastener deployment opening 404 while remaining insufficient to substantially deform the suture fastener 114 or otherwise interfere with subsequent deployment operations. Thus, as indexing mechanism 330 retracts rail 320, suture fastener 114 is retained within suture fastener deployment opening 404.

Once suture fastener 114 is appropriately positioned within suture fastener deployment opening 404, a suture can be introduced. The suture is introduced through side suture slot 116. The suture passes through side suture slot 116 and into suture fastener 114 via suture fastener opening 208. The trailing ends of the suture can exit distal tip 110 by extending through the curved portion of side suture slot 116. In some embodiments, side suture slot 116 can include one or more guide surfaces, ramps, funnels, chamfers, curved surfaces, retention features, or combinations thereof configured to direct the suture towards suture fastener opening 208 of suture fastener 114.

After the suture is captured within suture fastener 114, crimping jaws 112 can impart a crimping force upon suture fastener 114. Additional details regarding the function of crimping jaws 112 is provided below in the description of FIGS. 9A-B. The off-center configuration of suture fastener deployment opening 404 with respect to distal end face 402 allows for off-center crimping forces, which can reduce potential deformation of distal tip 110. Further, reinforced region 406 can be selected for appropriate material properties to withstand the crimping force and also reduce potential deformation of distal tip 110. In one example embodiment, crimping jaws 112 impart about 60 lbs. of crimping force. In such an example, 17-4PH H900 stainless steel having material properties allowing it to withstand greater than 60 lbs. can be chosen as the material for reinforced region 406.

The configuration of distal tip 110 described allows for suture fastener 114 to be retained and positioned in a deployment position using a frictional or interference fit. Further the side suture slot 116 of distal tip 110 allows for an operator to easily capture a suture such that the suture is restricted from disengaging from suture fastener delivery device 100 without being appropriately secured by suture fastener 114. Additionally, the off-center orientation of suture fastener deployment opening 404 and the material properties of reinforced region 406 allow for less potential for deformation due to repeated crimp forces.

Referring to FIG. 9, crimping mechanism 500 of suture fastener delivery device 100 is illustrated according to one embodiment of the present disclosure. Crimping mechanism 500 includes crimping jaws 112, hinge 502, jaw cam 504, and one or more biasing members 506. Crimping jaws 112 include floating jaw 508 and stationary jaw 510. Floating jaw 508 includes floating jaw crimping face 512 and cam contact face 514. Stationary jaw 510 includes stationary jaw crimping face 518. Jaw cam 504 includes jaw contact face 520.

Jaw cam 504 is operably coupled to the control element 108. Floating jaw 508 is coupled to hinge 502. Biasing members 506 are couple to hinge 502 and to floating jaw 508. Floating jaw 508 is positioned opposite stationary jaw 510 such that floating jaw crimping face 512 and stationary jaw crimping face 518 face each other. Crimping jaws 112 are positioned adjacent to fastener delivery end 106. Jaw cam 504 is positioned behind crimping jaws 112 with respect to fastener delivery end 106 such that jaw cam 504 extends toward shaft 104.

In operation, before actuation of crimping mechanism 500, the starting position of jaw cam 504 is such that the jaw contact face 520 is positioned adjacent to hinge 502. Further, floating jaw 508 is biased by biasing members 506 to be in a resting position. In the resting position, there exists a gap suitable for fitting suture fastener 114 between floating jaw crimping face 512 and stationary jaw crimping face 518, without any crimping force being imparted upon suture fastener 114. In one embodiment, biasing members 506 are nitinol wires extending between the floating jaw 508 and a portion of the distal tip 110.

In operation, upon activation of control mechanism 108, jaw cam 504 is actuated in a distal direction. Jaw contact face 520 is correspondingly moved distally and hence contacts cam contact face 514 of floating jaw 508. In response, biasing members 506 are elastically bent, and floating jaw crimping face 512 is pushed to pivot about hinge 502 and to close the gap between floating jaw crimping face 512 and stationary jaw crimping face 518. Accordingly, suture fastener 114 resting between floating jaw crimping face 512 and stationary jaw crimping face 518 is crimped by the force imparted from floating jaw 508. Once actuation is complete, jaw cam 504 retracts to its original starting position and floating jaw 508 is once again biased to its starting position via biasing members 506.

In some embodiments, jaw contact face 520 of jaw cam 504 and cam contact face 514 of floating jaw 508 are both corresponding angled surfaces such that the actuation load imparted from jaw cam 504 to floating jaw 508 is distributed over the contact surface area, thereby reducing localized stress on both jaw cam 504 and floating jaw 508. In some embodiments, jaw cam 504 comprises a polymeric component, such as an engineered plastic, optionally reinforced with glass fibers or other reinforcing materials to increase durability while maintaining desirable lubricity characteristics. As described in the description of FIG. 8, distal tip 110 may comprise a metallic material, including stainless steel, configured to resist deformation resulting from repeated crimping operations.

Crimping mechanism 500 provides several advantages. Floating jaw 508 permits controlled deformation of a suture fastener while reducing stresses imparted to distal tip 110 of suture fastener delivery device 100. Additionally, biasing members 506 facilitate the reliable return of floating jaw 508 to a resting position following a crimping operation. Further, the corresponding angled contact surfaces of jaw cam 504 and floating jaw 508 improve load distribution and durability during repeated crimping cycles.

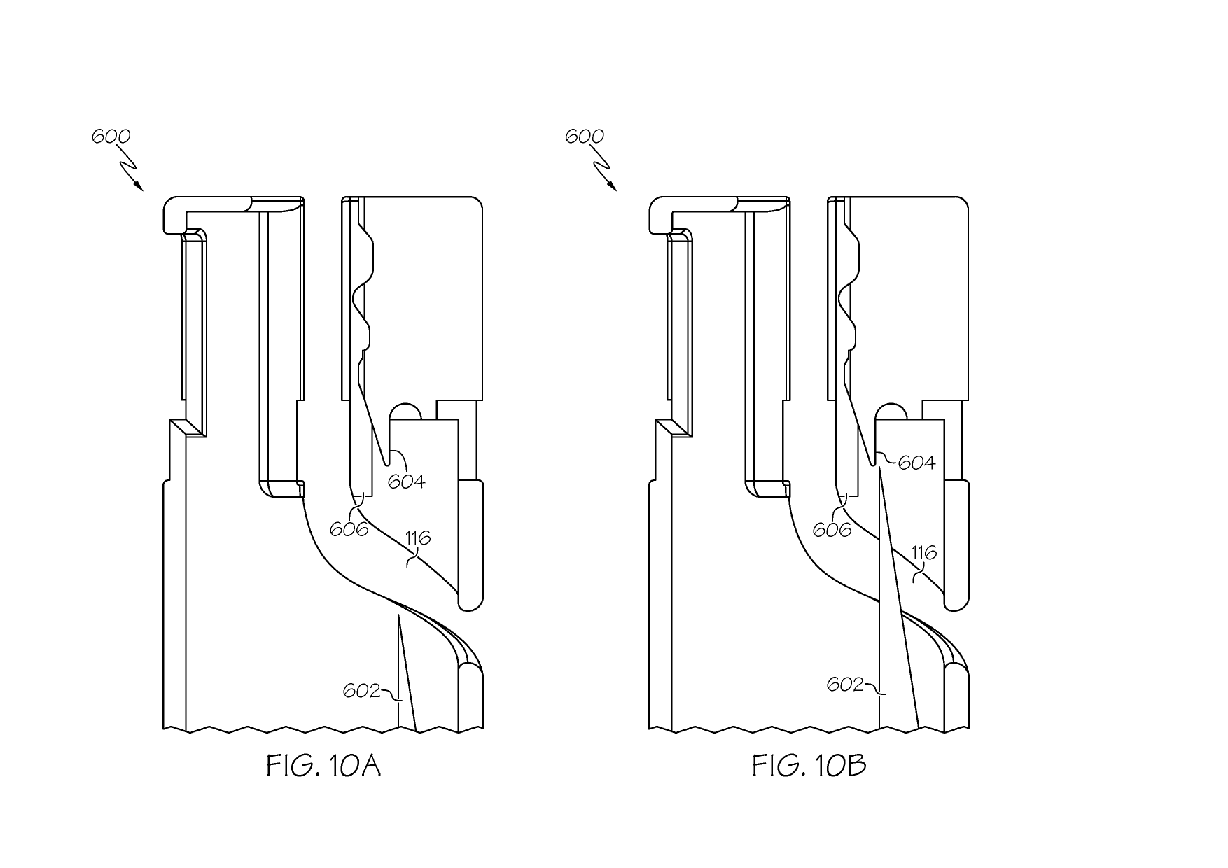

Referring to FIGS. 10A and 10B, cutting mechanism 600 of suture fastener delivery device 100 is illustrated according to one embodiment of the present disclosure. Cutting mechanism 600 includes first blade 602, second blade 604, and guide surface 606 positioned within distal tip 110.

In operation, before actuation of cutting mechanism 600, first blade 602 and second blade 604 are positioned on opposite sides of side suture slot 116. As described in the description of FIG. 8, a suture can be captured such that it is positioned within side suture slot 116 and within suture fastener 114. The suture passes over guide surface 606, thereby positioning the suture for more effective suture cutting. In some embodiments, guide surface is a curved surface. Based on the positioning of first blade 602 and second blade 604 and the position of guide surface 606, a suture passing through side suture slot 116 cannot unintentionally contact either of first cutting blade 602 and second cutting blade 604 during loading, positioning, indexing, or crimping operations.

In operation, upon actuation of cutting mechanism 600, first blade 602 is pushed in a proximal direction toward fastener delivery end 106. Accordingly, the suture passing through side suture slot 116 is severed by a scissor-like cut between first blade 602 and second blade 604. The suture is optimally positioned for such a cut to take place due to the suture passing over guide surface 606.

This disclosure contemplates various embodiments of first blade 602 and second blade 604. In some embodiments, both first blade 602 and second blade 604 provide a sharp edge, and first blade 602 is actuated toward second blade 604 to effectuate the cutting of the suture. In another embodiment, first blade 602 comprises a round cutting edge and first blade 602 is actuated toward second blade 604. In another embodiment, first blade 602 comprises a blunt face while second blade 604 comprises a sharp blade, and first blade 602 is actuated toward second blade 604.

Cutting mechanism 600 provides several advantages. The configuration of first blade 602, second blade 604, and guide surface 606 reduces the risk of premature suture cutting, before appropriate actuation of first blade 602. Additionally, guide surface 606 allows for optimally positioning and pulling taut a suture for cutting by cutting mechanism 600. Further, the scissor-like cutting structure of cutting mechanism 600 allows for less wear on the cutting blades as opposed to, for example, a guillotine arrangement which may result in more rapidly dulling or deformed blades.

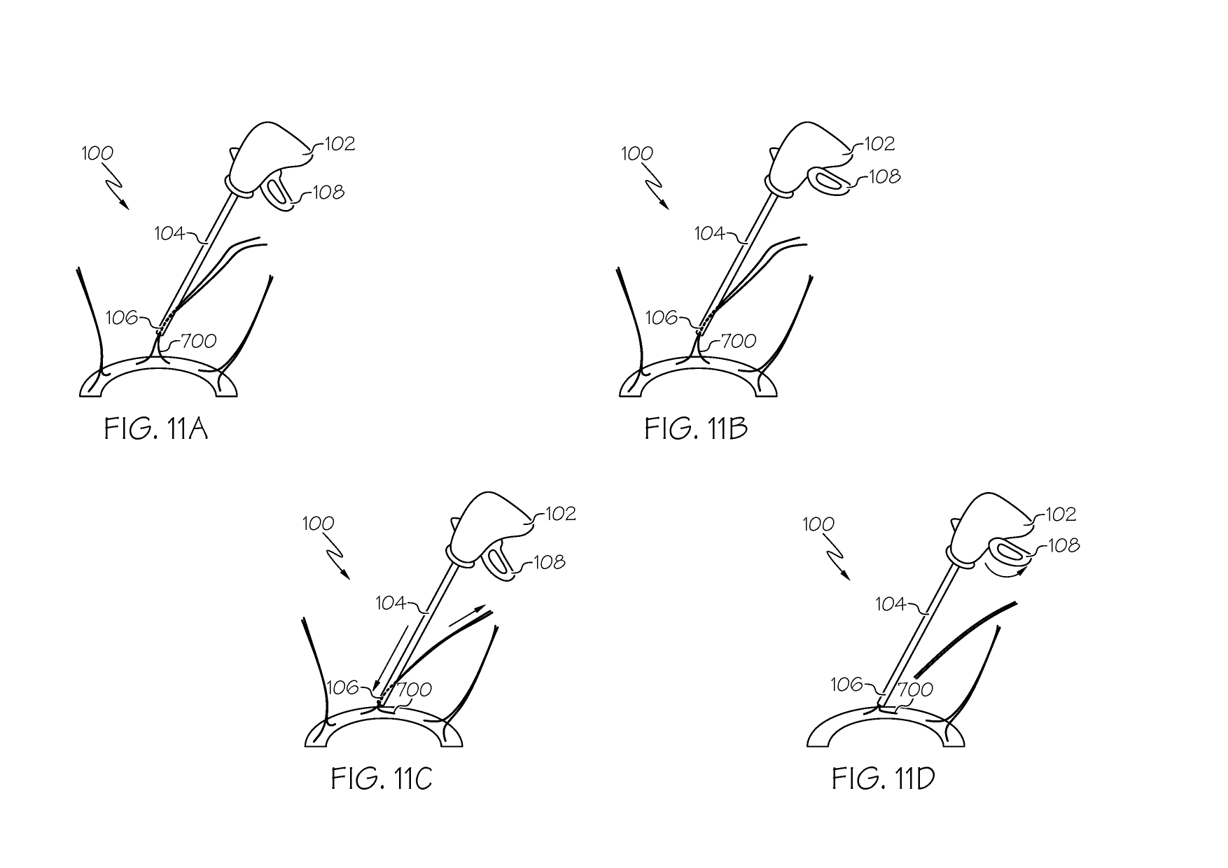

Referring to FIGS. 11A-11D, one embodiment of a method of deploying suture fastener 114 using suture fastener delivery device 100 is illustrated. The illustrated sequence demonstrates loading of a suture into suture fastener 114, pre-crimping of suture fastener 114, positioning of suture fastener 114 at a desired fixation location, and final crimping and cutting of the suture.

Referring to FIG. 11A, fastener delivery end 106 is positioned adjacent to a target suture 700. As discussed above, side suture slot 116 permits lateral introduction of suture 700 into distal tip 110 and into suture receiving opening 208 of suture fastener 114. Further as discussed above, trailing ends of suture 700 can extend through the curved portion of side suture slot 116. In the illustrated embodiment, suture fastener 114 has previously been advanced into a deployment position by indexing mechanism 330 and is retained in its undeformed state within distal tip 110 by crimping jaws 112. A user may position fastener delivery end 106 adjacent to suture 700 and introduce suture 700 into side suture slot 116 without requiring a free end of suture 700 to be threaded through suture fastener 114.

Referring to FIG. 11B, after suture 700 has been positioned within suture fastener 114, control element 108 may be actuated to place suture fastener 114 into a pre-crimped configuration. During the pre-crimping operation, crimping mechanism 500 partially deforms suture fastener 114 such that suture receiving opening 208 of suture fastener 114 is reduced in size, or closed, and suture 700 becomes captured within suture body 202. In the partially crimped configuration, suture 700 is substantially prevented from exiting suture fastener 114 through suture receiving opening 208 while remaining movable relative to suture fastener 114 along a longitudinal direction of suture 700. Accordingly, the partially crimped configuration secures suture 700 against accidental disengagement while preserving the ability to reposition suture fastener 114.

Referring to FIG. 11C, with suture fastener 114 maintained in the partially crimped configuration, an operator may translate suture fastener delivery device 100 to position fastener delivery end 106, and hence suture fastener 114, to a desired fastening location. Such translation is accomplished by sliding suture fastener 114 along suture 700 until the desired fastening location is reached. The trailing ends of suture 700 continue to be fed through the curved portion of side suture slot 116.

Referring to FIG. 11D, after suture fastener 114 has been positioned at a desired location, control element 108 may be further actuated to place suture fastener 114 into a fully crimped configuration and to cut the trailing suture ends of suture 700. During the final crimping operation, crimping mechanism 500 further deforms suture fastener 114 such that suture 700 is securely retained within suture body 202. Immediately after or simultaneous with crimping, cutting mechanism 600 is activated to sever a trailing portion of suture 700. Following completion of the final crimping and cutting operations, suture fastener 114 remains secured to suture 300 at the desired fastening location and excess suture material may be removed from the surgical site.

The sequence illustrated in FIGS. 11A-11D provides several advantages. The pre-crimped configuration permits capture of suture 700 while maintaining the ability to reposition and tension the suture prior to final fixation. Additionally, loading, pre-crimping, final crimping, and cutting may all be performed using a single delivery device 100 and using a single control mechanism 108, thereby reducing procedural complexity and improving consistency of fastener deployment.

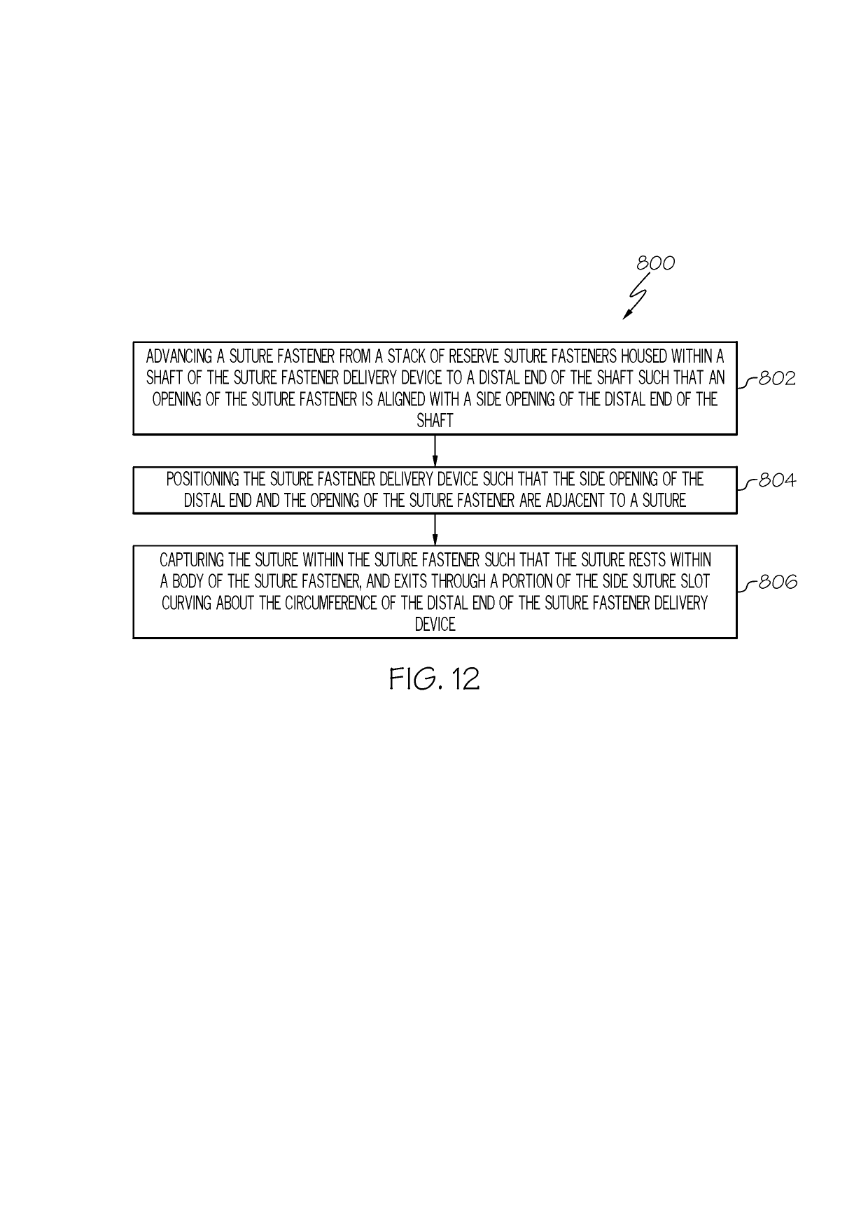

Referring to FIG. 12, one embodiment of method 800 for loading a suture into suture fastener 114 using suture fastener delivery device 100 is illustrated. Method 800 begins at step 802. At step 802, suture fastener 114 is advanced from stack of reserve suture fasteners 322 housed within shaft 104 of suture fastener delivery device 100 to fastener delivery end 106. As discussed above, indexing mechanism 330 advances suture fastener 114 to a deployment position such that suture receiving opening 208 of suture fastener 114 is aligned with side suture slot 116 of distal tip 110. In some embodiments, advancing the suture fastener 114 comprises actuating control element 108 to a first actuation position.

At step 804, suture fastener delivery device 100 is positioned such that fastener delivery end 106 is placed adjacent to a target suture, such as suture 700.

At step 806, a user may then introduce suture 700 into side suture slot 116 such that suture 700 enters suture receiving opening 208 of suture fastener 114. Suture 700 thus rests within body 202 of suture fastener 114 and trailing portions of suture 700 extend outwardly through side suture slot 116, including a curved portion thereof extending about at least a portion of the circumference of distal tip 110. Accordingly, method 800 allows for capturing suture 700 securely within suture fastener delivery device 100.

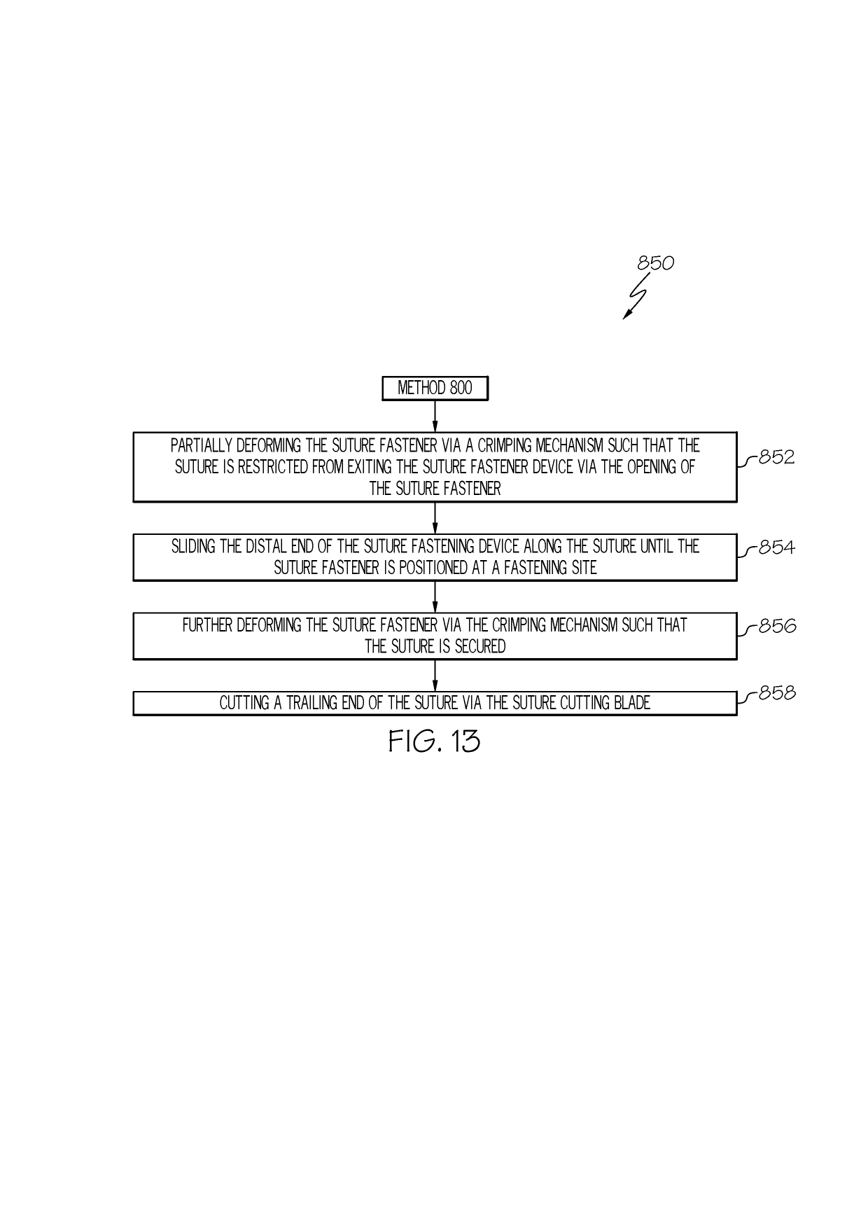

Referring to FIG. 13, one embodiment of method 850 for securing suture 700 using suture fastener delivery device 100 is illustrated. Method 850 can be executed after the execution of method 800, as described above.

At step 852, after suture 700 has been positioned within suture fastener 114, crimping mechanism 500 may be actuated to partially deform suture fastener 114. Partial deformation of suture fastener 114 reduces the size of suture receiving opening 208 such that suture 700 is restricted from exiting suture fastener 114 through suture receiving opening 208 while remaining movable relative to the suture fastener 114. In some embodiments, suture fastener 114 is deformed into a tubular shape, such that suture receiving opening 208 is closed. In some embodiments, partially deforming suture fastener 114 comprises actuating control element 108 to a second actuation position.

At step 854, distal tip 110 can be translated along suture 700 until suture fastener 114 reaches a desired fastening location within a surgical field.

At step 856, crimping mechanism 500 can be further actuated to further deform suture fastener 114 and securely retain suture 700 within the suture fastener 114. In some embodiments, further actuating crimping mechanism 500 comprises actuating control element 108 to a third actuation position.

At step 858, cutting mechanism 600 is actuated to sever a trailing portion of suture 700 via suture cutting blade 602. In some embodiments, actuating cutting mechanism 600 comprises actuating control element 108 to the third actuation position. In such an embodiment, the cutting of the trailing portion of suture ends 700 can occur simultaneous to, or immediately after, the further deformation of suture fastener 114.

Accordingly, method 850 allows for suture fastener delivery device 100 to capture, reposition, secure, and trim suture 700 using a single instrument and a single suture fastener 114.

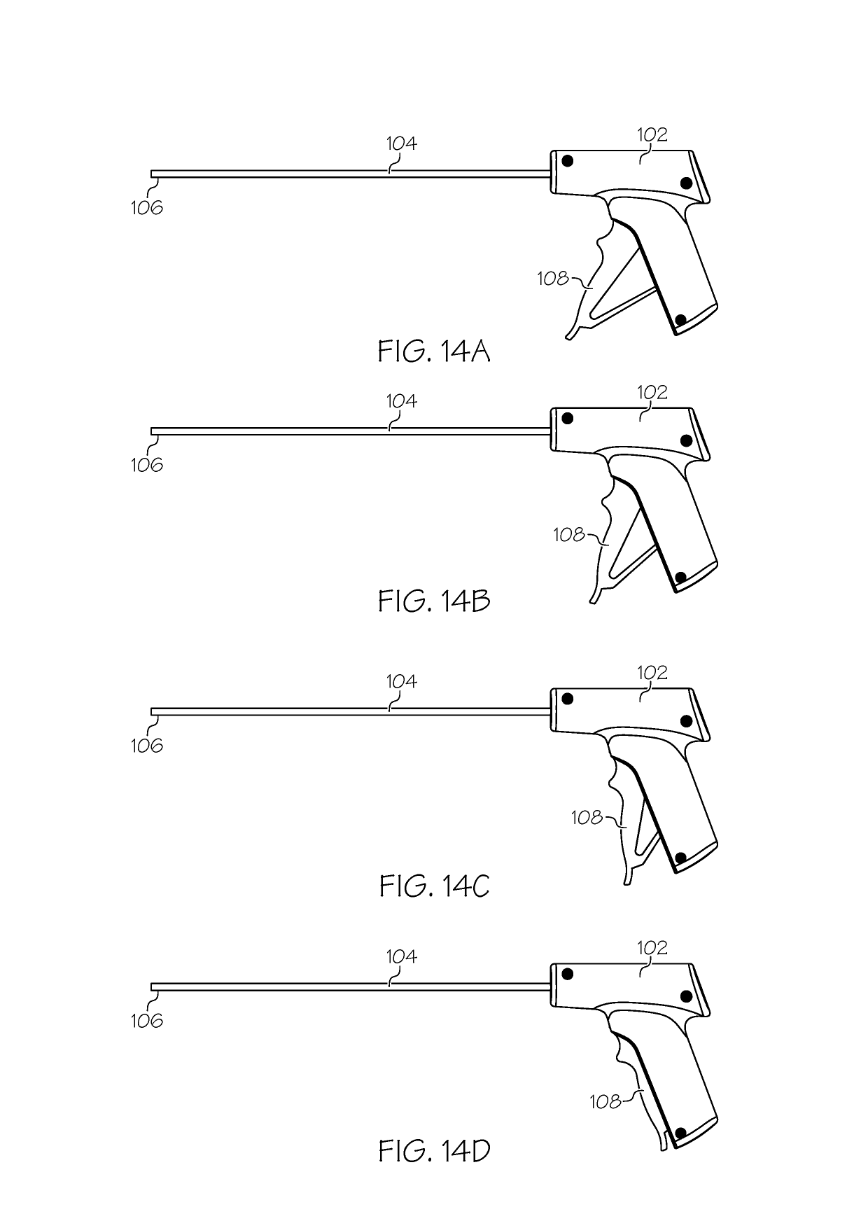

Referring to FIGS. 14A-14D, one embodiment of the operation of control element 108 is illustrated. In the illustrated embodiment, control element 108 is configured to actuate multiple functions of suture fastener delivery device 100 through a sequence of discrete actuation positions. More particularly, control element 108 may be selectively advanced from a starting position, to a fastener reloading position, to a partial crimping position, and finally to a full crimping and cutting position. In certain embodiments, each actuation position corresponds to a distinct operational stage of the suture fastener delivery device 100.While control element 108 is depicted as a trigger, it is understood that any suitable control mechanism may be substituted (e.g., button, lever, slider, knob, etc.)

Referring to FIG. 14A, control element 108 is illustrated in a starting position. In the starting position, indexing mechanism 330, crimping mechanism 500, and cutting mechanism 600 are maintained in respective resting states. Reserve suture fasteners 322 are arranged in stack along shaft 104. Distal tip 110 is not populated with a suture fastener.

Referring to FIG. 14B, control element 108 is actuated to a first actuation position corresponding to a fastener reloading operation. During the reloading operation, indexing mechanism 330 advances a reserve suture fastener 114 toward fastener delivery end 106 and positions the suture fastener 114 for deployment. In certain embodiments, advancement of control element 108 to the first actuation position causes the control element 108 to engage a first stop position corresponding to completion of the reloading operation.

Referring to FIG. 14C, control element 108 is actuated to a second actuation position corresponding to a partial crimping operation. During the partial crimping operation, crimping mechanism 330 partially deforms suture fastener 114 such that a suture received within the suture fastener 114 is substantially prevented from escaping through suture receiving opening 208 while remaining movable relative to the suture fastener 114. In certain embodiments, advancement of control element 108 to the second actuation position causes the control element 108 to engage a second stop position corresponding to completion of the partial crimping operation.

Referring to FIG. 14D, control element 108 is actuated to a third actuation position corresponding to a final crimping and cutting operation. During the final crimping operation, crimping mechanism 500 further deforms suture fastener 114 to securely retain the suture within the suture fastener 114. During the cutting operation, control element 108 actuates cutting mechanism 600 to sever excess suture material. In some embodiments, the final crimping operation and the cutting operation occur simultaneously. In other embodiments, the crimping operation occurs first, followed by the cutting operation. Following completion of the crimping and cutting operation, suture fastener 114 remains secured to the suture and suture fastener delivery device 100 may be prepared for a subsequent deployment cycle.

In some embodiments, advancing from one position to the next position requires the user to release control mechanism 108. Thus, for example, after actuating control mechanism 108 from the starting position of FIG. 14A to the fastener reloading position of FIG. 14B, the user must release control mechanism 108, before reapplying pressure to control mechanism 108 to move to the pre-crimping position of FIG. 14C. In some embodiments, an audible or tactile click occurs when actuating the control element 108 from one position to the next position (e.g., from the starting position to the fastener loading position) to signal the user that the next position has been reached.

Control mechanism 108 provides several advantages. A single control mechanism is used to perform fastener reloading, pre-crimping, final crimping, and suture cutting operations, thereby simplifying the user inputs required during a surgical procedure. Additionally, the discrete actuation positions and associated stop positions provide tactile feedback to the user regarding completion of each operational stage.

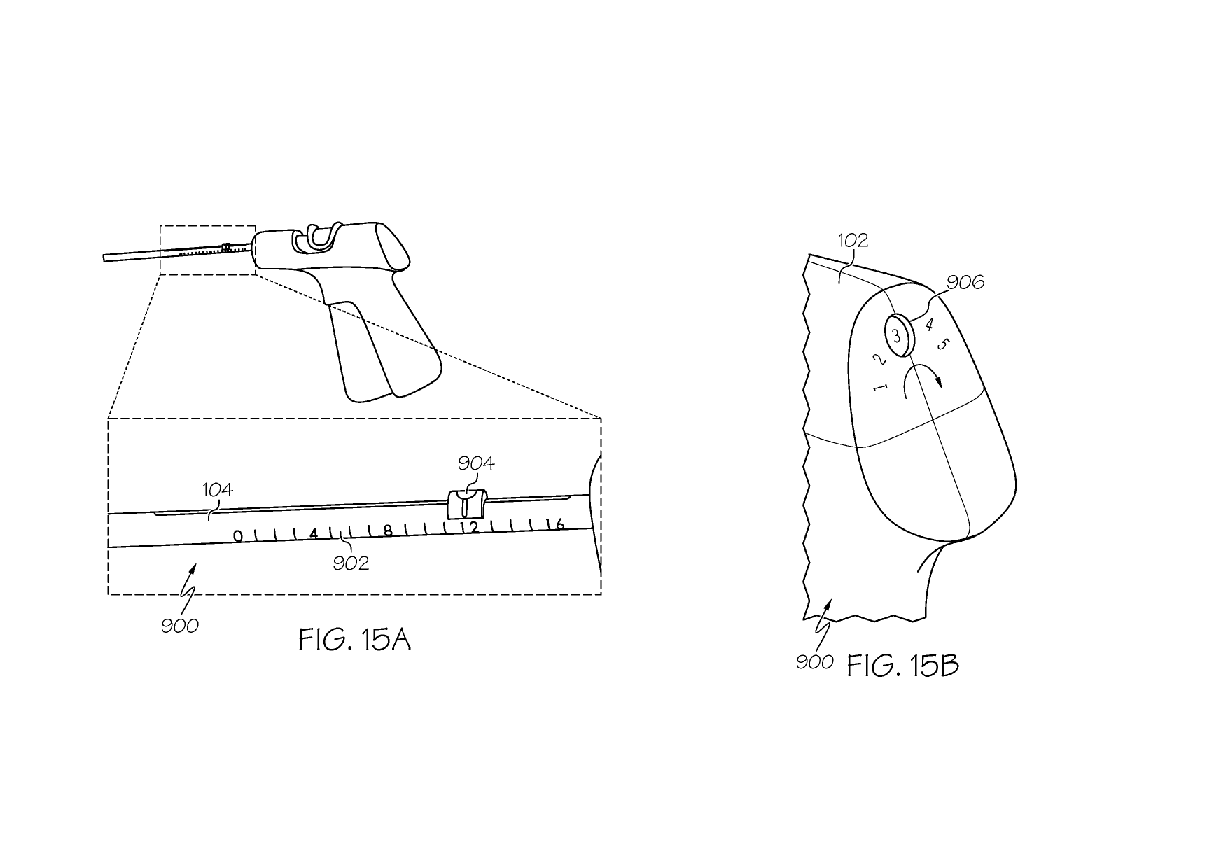

Referring to FIGS. 15A and 15B, a fastener counting mechanism 900 is illustrated according to various embodiments of the present disclosure. Counting mechanism 900 is configured to provide a visual indication of the quantity of reserve suture fasteners 322 remaining within suture fastener delivery device 100. In certain embodiments, counting mechanism 900 is operably coupled to indexing mechanism 330 such that advancement and deployment of individual suture fasteners 114 causes a corresponding change in the displayed count.

FIG. 15A depicts counting mechanism 900 as being positioned along shaft 104 of suture fastener delivery device 100. In the illustrated embodiment, one or more indicia 902 are provided along an exterior surface of shaft 104 and correspond to the quantity of reserve suture fasteners 322 remaining within the device. A counter indicator 904 is movable relative to indicia 902 and is configured to identify the current quantity of reserve suture fasteners 322 available for deployment. In certain embodiments, indicia 902 comprise numerical markings corresponding to a quantity of remaining suture fasteners 114.

FIG. 15B depicts counting mechanism 900 as being positioned on controller 102. In the illustrated embodiment, controller 102 includes a viewing region 906 configured to display a remaining fastener count. The viewing region 906 may include a rotatable wheel, movable strip, sliding indicator, dial, numerical display, or other suitable indicator mechanism configured to communicate the quantity of reserve suture fasteners 322 remaining within the device. In certain embodiments, deployment of each suture fastener 114 causes the displayed count to decrease by one.

Counting mechanism 900 advantageously provides a user with immediate feedback regarding the availability of additional suture fasteners 114.

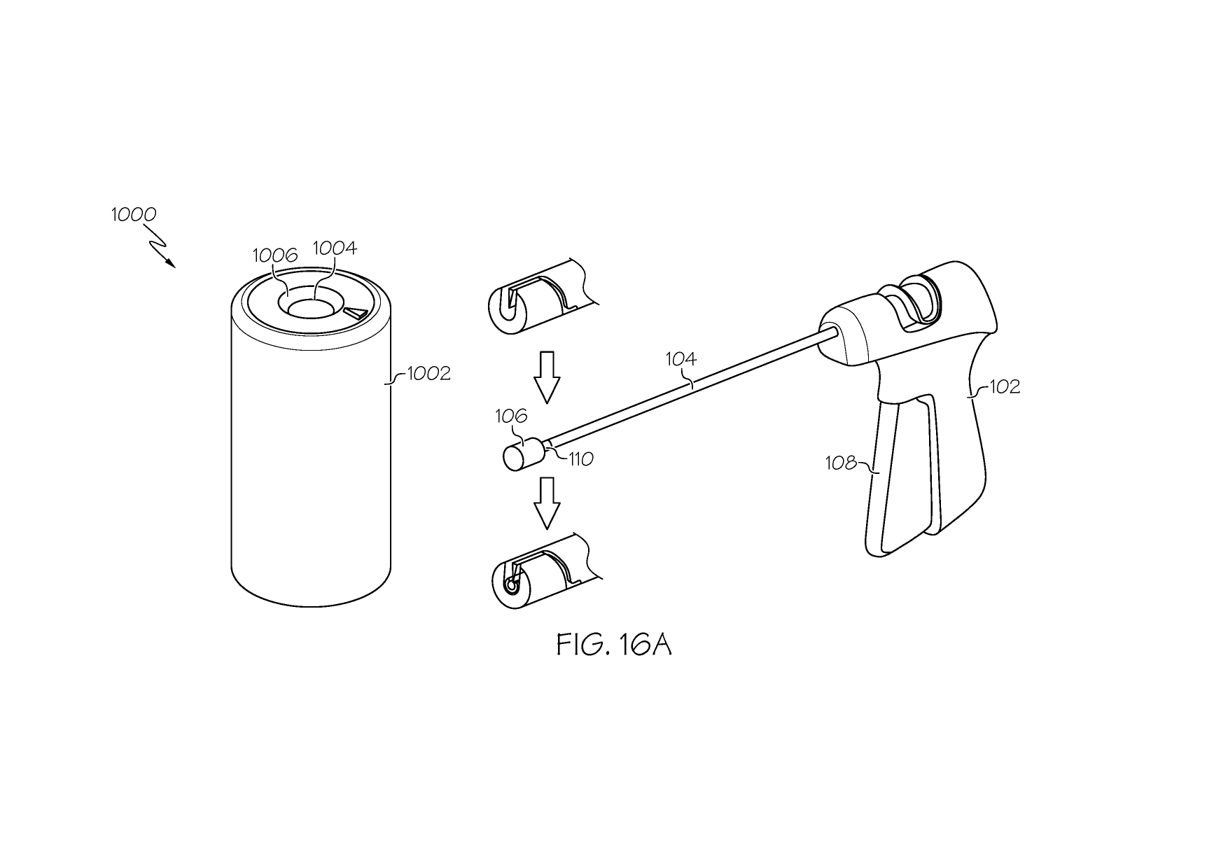

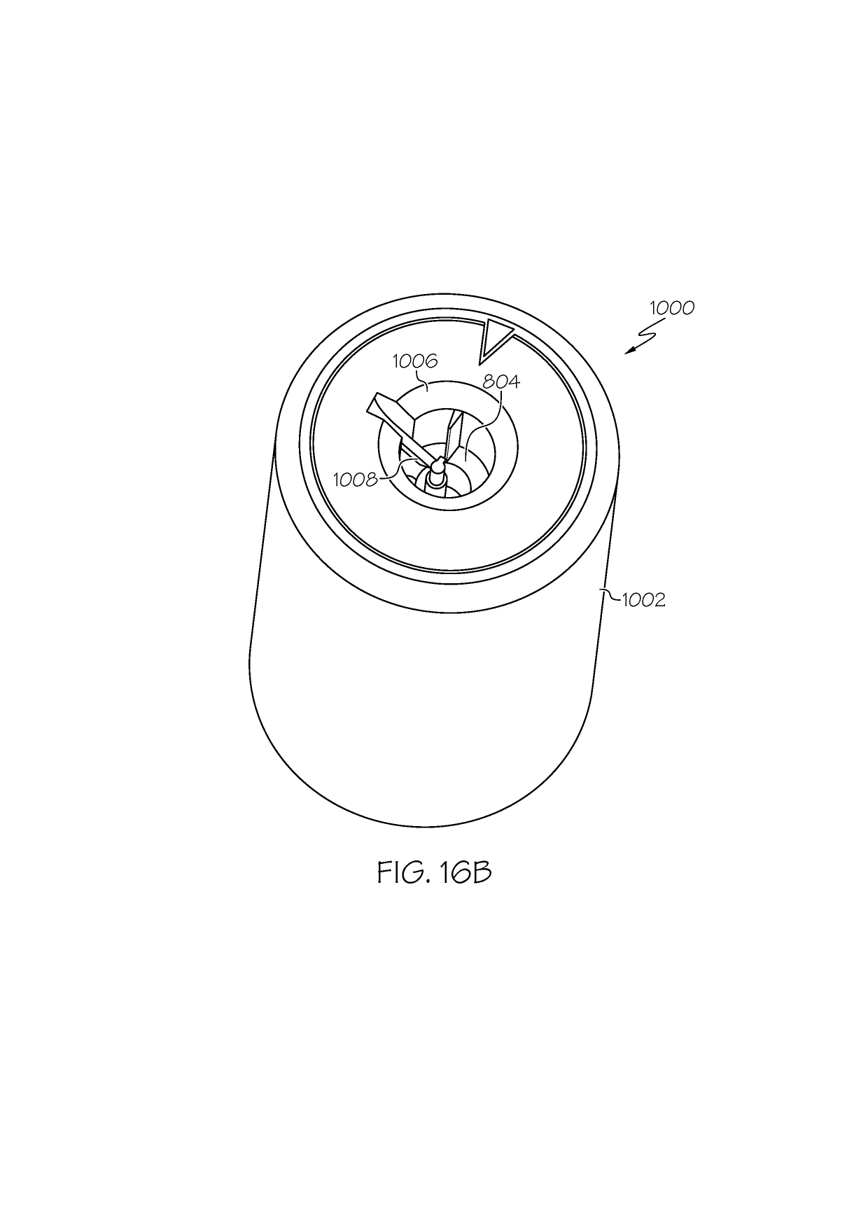

Referring to FIGS. 16A and 16B, a single-use loader 1000 is illustrated according to one embodiment of the present disclosure. Single-use loader 1000 is configured to facilitate loading of an individual suture fastener 114 into fastener delivery end 106 of suture fastener delivery device 100. In the illustrated embodiment, single-use loader 1000 includes a loader body 1002 defining an internal cavity 1004 configured to receive and support a suture fastener 114 and flexible retainer 1008 configured to retain suture fastener within internal cavity 1004. Loader body 1002 further includes an insertion opening 1006 configured to receive at least a portion of distal tip 110 of suture fastener delivery device 100. Single-use loader 1000 is supplied with a suture fastener 114 preloaded within internal cavity 1004 such that the single-use loader 1000 may be utilized to load a single suture fastener within suture fastener delivery device 100.

As best illustrated in FIG. 16A, distal tip 110 may be inserted into insertion opening 1006 such that distal tip 110 becomes aligned with suture fastener 114 positioned within internal cavity 1004. Continued insertion of distal tip 110 causes flexible retainer 1008 (as shown in FIG. 16B) to be displaced and hence suture fastener 114 to be transferred from single-use loader 1000 into distal tip 110. In certain embodiments, insertion of distal tip 110 into single-use loader 1000 produces an audible, tactile, or visual indication confirming that suture fastener 114 has been properly positioned within fastener delivery end 106. Following transfer of suture fastener 114, single-use loader 1000 may be removed from distal tip 110 and discarded.

Single-use loader 1000 provides several advantages. Single-use loader 1000 permits an individual suture fastener 114 to be loaded into suture fastener delivery device 100 without the need for reloading the entire device. Thus, in the surgical field where only a single or a few additional suture fasteners are needed after exhausting reserve suture fasteners 322 in shaft 104, single-use loader 1000 can be used. Additionally, flexible retainer 1008 ensures that during shipping or handling of single-use loader 1000, suture fastener 114 does not inadvertently fall out of or otherwise become displaced from single-use loader 1000 while also permitting rapid loading when additional suture fasteners are needed.

According to the techniques of this disclosure, a suture fastener delivery device is provided that facilitates the deployment of suture fasteners to secure sutures within a surgical field. The techniques of this disclosure also relate to improvements to the suture fastener, including discontinuous collar configurations, suture retention features, suture positioning features, and frictional elements configured to improve retention of a suture following deployment. The techniques of this disclosure further relate to improved indexing mechanisms for sequential advancement of reserve suture fasteners, crimping mechanisms including floating jaws, jaw cams, and resilient return members, and cutting mechanisms configured to provide reliable severing of excess suture material.

The disclosure additionally describes methods of operating the delivery device, including loading a suture into a suture fastener, pre-crimping the suture fastener to capture the suture while permitting repositioning, positioning the suture fastener at a desired fastening location, and subsequently fully crimping the suture fastener and cutting excess suture material. Further disclosed are control mechanisms configured to sequentially actuate reloading, partial crimping, full crimping, and cutting operations, counting mechanisms configured to indicate a quantity of remaining reserve suture fasteners, and single-use loading devices configured to facilitate loading of additional suture fasteners into the delivery device.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof.

As used herein any reference to "one embodiment" or "an embodiment" means that a particular element, feature, structure, or characteristic described in connection with the embodiment is included in at least one embodiment. The appearances of the phrase "in one embodiment" in various places in the specification are not necessarily all referring to the same embodiment.

As used herein, the terms "comprises," "comprising," "includes," "including," "has," "having" or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, "or" refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the "a" or "an" are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments for purposes of illustration only. One skilled in the art will readily recognize from the discussion herein that alternative embodiments of the structures and methods illustrated herein may be employed without departing from the principles described herein.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

While the systems and methods described herein have been described in reference to some exemplary embodiments, these embodiments are not limiting and are not necessarily exclusive of each other, and it is contemplated that particular features of various embodiments may be omitted or combined for use with features of other embodiments while remaining within the scope of the invention.

1. A suture fastener delivery device, comprising:
a controller positioned on a proximal end of the suture fastener delivery device, the controller comprising a control element;
a fastener delivery end positioned on a distal end of the suture fastener delivery device, the fastener delivery end comprising:
a suture fastener comprising a suture receiving opening configured to be deployed to secure a suture;
crimping jaws positioned about an outer surface of the suture fastener;
a shaft extending between the controller and the fastener delivery end from the proximal end to the distal end of the suture fastener delivery device, the shaft comprising:
a rail extending along an interior length of the shaft;
one or more reserve suture fasteners mounted along the rail;
a side suture slot positioned about a side of the shaft and aligned with the suture receiving opening of the suture fastener, the side suture slot configured to receive the suture and to allow trailing ends of the suture to exit the side suture slot; and
a suture cutting blade, the suture cutting blade positioned adjacent to the rail within the shaft.

2. The suture fastener delivery device of claim 1, wherein the suture fastener has a substantially C-shaped cross section and comprises a collar extending circumferentially about an outer surface of the suture fastener adjacent a first end thereof.

3. The suture fastener delivery device of claim 2, wherein the collar comprises a discontinuity, the discontinuity comprising a gap extending through the collar.

4. The suture fastener delivery device of claim 3, wherein the discontinuity is positioned opposite the suture receiving opening.

5. The suture fastener delivery device of claim 2, wherein the collar comprises a protrusion extending into the suture receiving opening, the protrusion configured to retain the suture within the suture fastener.

6. The suture fastener delivery device of claim 2, wherein the collar comprises a groove, the groove configured to push the suture to a preferred position within the suture fastener.

7. The suture fastener delivery device of claim 6, wherein the groove is a partial indentation in the collar and where the groove is positioned adjacent to the suture receiving opening.

8. The suture fastener delivery device of claim 1, wherein an inner diameter of the suture fastener includes one or more frictional elements configured to increase retention of the suture after crimping.

9. The suture fastener delivery device of claim 1, wherein actuation of the control element to a first actuation position advances a distal-most reserve suture fastener to the fastener delivery end.

10. The suture fastener delivery device of claim 1, wherein the fastener delivery end comprises a distal tip, and wherein the crimping jaws impart a crimping force at a location offset from a central axis of the distal tip.

11. The suture fastener delivery device of claim 1, wherein the crimping jaws include a floating jaw positioned within the shaft and adjacent to the fastener delivery end.

12. The suture fastener delivery device of claim 11, further comprising a jaw cam housed within the shaft, the jaw cam comprising a contact surface and the jaw cam configured to engage with the floating jaw at the contact surface to impart a crimping force.

13. The suture fastener delivery device of claim 12, wherein the contact surface is angled relative to a direction of movement of the jaw cam.

14. A method for installing a suture fastener within a surgical field via a suture fastener delivery device, the method comprising
advancing a suture fastener from one or more reserve suture fasteners housed within a shaft of the suture fastener delivery device to a distal end of the shaft such that an opening of the suture fastener is aligned with a side opening of the distal end of the shaft;
positioning the suture fastener delivery device such that the side opening of the distal end and the opening of the suture fastener are adjacent to a suture; and
capturing the suture within the suture fastener such that the suture rests within a body of the suture fastener, and exits through a portion of the side suture slot curving about the circumference of the distal end of the suture fastener delivery device.

15. The method of claim 14, further comprising:
partially deforming the suture fastener via a crimping mechanism such that the suture is restricted from exiting the suture fastener device via the opening of the suture fastener;
sliding the distal end of the suture fastening device along the suture until the suture fastener is positioned at a fastening site;
further deforming the suture fastener via the crimping mechanism such that the suture is secured; and
cutting a trailing end of the suture via the suture cutting blade.

16. The method of claim 15, wherein advancing the suture fastener comprises actuating a control element to a first actuation position.

17. The method of claim 16, wherein partially deforming the suture fastener comprises actuating the control element to a second actuation position.

18. The method of claim 17, wherein further deforming the suture fastener comprises actuating the control element to a third actuation position.

19. The method of claim 15, wherein a control element is movable between a plurality of actuation positions, each actuation position corresponding to a different operational stage of the suture fastener delivery device, and wherein the control element engages a stop position at each actuation position thereby providing tactile feedback to a user.

20. The method of claim 15, wherein the suture fastener transitions from a C-shaped configuration to a closed tubular configuration after partial deformation.
